(19) United States
(12) Patent Application Publication
(10) Pub. No.: US 2003/0216341 A1
(43) Pub. Date: Nov. 20, 2003

(54) Multiple genes relevant for the characterization, diagnosis, and manipulation of neuropathic pain

(76) Inventors: Hermann Lubbert, Leverkusen (DE); Peter Engels, Bergisch Gladbach (DE); Beate Schmitz, Koln (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/368,819

(22) Filed: Feb. 14, 2003

Related U.S. Application Data
(60) Provisional application No. 60/357,744, filed on Feb. 14, 2002.

(51) Int. Cl. A61K 48/00; C12Q 1/68

(52) U.S. Cl. 514/44; 435/6

(57) ABSTRACT

The present invention relates to the use of differentially expressed polynucleotide sequences or polypeptides for the characterization of pain status or progression, a method for characterizing pain, a method for identifying therapeutic agents for pain and the use of such sequences for the development of a medicament.

### RELATED APPLICATION

This application claims priority under 35 U.S.C. §119(e) to U.S. Provisional Application No. 60/357,744, filed Feb. 14, 2002.

### FIELD OF THE INVENTION

The present invention relates to the use of differentially expressed polynucleotide sequences or polypeptides for the characterization of pain status or progression, a method for characterizing pain, a method for identifying therapeutic agents for pain and the use of such sequences for the development of a medicament.

### DESCRIPTION OF THE RELATED ART

The effective treatment of pain requires an understanding of its physiology. It is well known, however, that stimuli, which activate pain receptors in one tissue, may not activate pain receptors in another. For example, pricking or cutting, which causes pain in skin tissue, does not cause pain in the stomach or intestine. The causes of pain in skeletal muscle, joints, and arteries can also differ (Principles of Neurology, 6^{th} ed. Adams, R. D. et al. eds. McGraw-Hill: 1997 pp. 133-134). Consequently, methods useful for relieving one type of pain are often less effective, or even ineffective, when applied to the alleviation of others. In general, neuropathic pain is persistent and is characterized by burning, gnawing, aching, shooting, or lancinating sensations. It is frequently associated with hyperesthesia, hyperalgesia, allodynia, and hyperpathia, and in some cases by sensory deficit or autonomic dysfunction. Unfortunately, and unlike other types of pain, neuropathic-pain tends to respond poorly to analgesic medication (Principles of Neurology, 6^{th} ed., Adams, R. D., et al. eds. McGraw-Hill 1997 p. 140).

Depending on the nerves involved, a particular instance of neuropathic pain can be classified as a central or peripheral neuropathy. Central neuropathies arise from spinal cord, brainstem, thalamic, and cerebral damage or disease, while peripheral neuropathies arise from damage or disease of peripheral nerves. Specific peripheral neuropathies include, but are not limited to: thoracic outlet obstruction syndromes; compression and entrapment neuropathies such as ulnar nerve palsy, carpal tunnel syndrome, peroneal nerve palsy, radial nerve palsy; and Guillain-Barré syndrome (The Merck Manual, 16^{th} ed. 1992 1518-1522).

Neuropathic, or neurogenic, pain arises from the direct stimulation of nervous tissue. Neuropathic pain encompasses a wide variety of disorders involving single and multiple nerves. These include, but are not limited to, trigeminal neuralgia and disorders due to herpes zoster, diabetes, and trauma (including causalgia); spinal arachnoiditis and spinal cord injuries; and the thalamic pain syndrome of Dejerine-Roussy (Principles of Neurology, 6^{th} ed., Adams, R. D. et al. ed. McGraw-Hill 1997 p. 140).

Neuropathic pain is caused by a variety of factors including, but not limited to: trauma caused by injury or surgical operation; tumors; bony hyperostosis; casts; crutches; prolonged cramped postures; hemorrhage into a nerve; exposure to cold or radiation; collagen-vascular disorders; infectious diseases such as Lyme disease, HIV; and Herpes zoster; toxins such as emetine, hexobarbital, barbital, chlorobutanol, sulfonamides, nitrofurantoin, the vinca alkaloids, heavy metals, carbon monoxide, triorthocresylphosphate, orthodinitrophenol, and other solvents and industrial poisons; autoimnune reactions; nutritional deficiency, and vitamin B deficiency in particular; and metabolic disorders such as hypothyroidism, porphyria, sarcoidosis, amyloidosis, uremia and diabetes (The Merck Manual, 16th ed. 1992 1518).

Because so many causes of neuropathic pain exist, and because it tends to respond poorly to analgesic medication, the discovery of drugs that safely and effectively aid in its relief has been difficult.

Whereas acute pain is generally symptomatic of tissue damage or inflammation, and lasts only as long as the underlying cause remains, chronic pain may persist indefinitely in the absence, or after recovery of tissue pathology. It may occur as the aftermath of injury (e.g., to peripheral nerves or to the spinal cord), or in association with other disease states, such as herpes zoster or diabetes, but often it arises with no obvious cause. Chronic pain is often severe, leading to major disability and a high suicide rate, and it is common, with a prevalence of approximately 1% at the severe level. In contrast to acute pain, chronic pain is often unresponsive to conventional analgesic drugs (opiates and NSAIDs), and presents a difficult therapeutic problem, since its cause can usually not be resolved. Various other classes of drugs, (e.g. tricyclic antidepressants, some anticonvulsant drugs, such as gabapentin, may be effective, but the therapeutic response is very variable and often accompanied with severe side effects.

A common feature of many clinical syndromes where chronic pain develops is the existence of previous nerve damage, affecting peripheral nerves, the spinal cord or (as in stroke) the brain, and the concept of neuropathic pain (i.e. pain arising as a consequence of neuronal damage) has become accepted as the underlying cause of many different chronic pain conditions seen in the clinic. Several animal models of neuropathic pain have been developed, which mimic many aspects of the clinical condition. These include lesions of the sciatic nerve (constriction or partial section), constriction or section of spinal nerves, ischemic lesions of the spinal cord, induction of diabetic neuropathy, etc., and such models have been subjected to detailed study of the anatomical, biochemical and physiological changes that accompany the development of the pain state.

In the last several years a number of experimental models for neuropathic pain have been developed (Bennett & Xie 1988 Pain 33:87-107; Seltzer et al. 1990 Pain 43:205-218; Kim & Chung 1992 Pain 50:355-363; DeLco et al. 1994 Pain 56:9-16; Na et al. 1994 Neurosci Lett 177:50-52). The availability of these different models provides an opportunity to investigate mechanisms of neuropathic pain. Finding common features in different models should provide better insight into the mechanisms critical for neuropathic pain, and comparison of the models should help to understand pain development and progression.

Kim et al. compared three of the models on basis of neuropathic pain behaviors and the effects of surgical sympathectomy (Kim et al. 1997 Exp Brain Res 113:200-206). They found that the models of Bennett, Seltzer and Kim & Chung (see above) result in a very similar general pattern and time course of evoked pain behavior, whereby the Bennett model showed biggest behavioral signs for ongoing pain. The same results have been shown for the effects of sympathectomy on the behavioral signs of evoked and ongoing neuropathic pain, respectively. Thus these three models can be used to discover basic common features involved in neuropathic pain.

Further animal models for pain are considered in the article of Walker et al. 1999 Molecular Medicine Today 5:319-321, comparing models for different types of pain, which are acute pain, chronic/inflammatory pain and chronic/neuropathic pain, on the basis of behavioral signs.

Object of the present invention is to identify and characterize development, conditions and progression of pain on the molecular basis.

### SUMMARY OF THE INVENTION

This object is met by the use of polynucleotide sequences selected from the group of sequences SEQ ID NO: 1 to 64 or homologues or fragments thereof or the according polypeptides for the characterization of a) the status which elicits pain and/or b) the progression of the pathology of pain, whereby the characterization is carried out outside of a living body.

Polynucleotide sequences SEQ ID NO: 1 to 64 are expressed sequence tags (ESTs) representing genes which are differentially expressed under pain, particularly under neuropathic pain, in the models of Bennett, Seltzer and Kim & Chung (Bennett & Xie 1988 Pain 33:87-107, Seltzer et al. 1990 Pain 43:205-218; Kim & Chung 1992 Pain 50:355-363).

In one embodiment a combination of at least four polynucleotide sequences selected from the group of sequences SEQ ID NO: 1 to 64 or homologues or fragments thereof or the according polypeptides is used for the characterization of (a) the status which elicits pain and/or (b) the progression of the pathology of pain. Such characterization is carried out outside of a living body. The sequences are preferably of mammalian origin.

In another embodiment a pain status is characterized by comparing of the expression of at least four genes comprising the sequences selected of the group of SEQ ID NO. 1 to 64 or homologues or fragments thereof to a pain-free status. In one embodiment such characterizing of the pain status is performed outside of the living body. In one preferred embodiment a pain status is characterized for assessing the efficacy of a pain treatment outside of a living body. In another preferred embodiment a pain status is characterized for assessing of animal models for pain.

In one preferred embodiment the expression is determined from in vivo samples, in vitro samples, or ex vivo samples. Such samples are derived from whole tissues, blood, cerebrospinal fluid (CSF), from cell populations isolated from tissues, blood or CSF or from cell lines.

In one preferred embodiment a combination of at least six sequences is compared to non-disease status.

In one preferred embodiment the considered gene expression is increased compared to a non-disease status. In another preferred embodiment the considered gene expression is decreased compared to a non-disease status. In yet another preferred embodiment the considered gene expression for at least one sequence is increased and for at least one sequence is decreased compared to a non-disease status.

In one preferred embodiment a method is provided comprising the steps of: a) providing a test sample comprising cells or body fluids expressing or containing one or more genes or gene products represented by polynucleotide sequences selected from the group of SEQ ID NO. 1 to 64 or homologues or fragments thereof; b) detecting expression of one or more of the genes in the sample; c) comparing the expression of the genes in the test sample to the expression of the same genes in reference samples whose expression stage is known, and d) identifying a difference in the expression levels of the considered sequences, if present, in the test sample and the reference sample.

In another preferred embodiment, a method of identifying a test therapeutic agent for treating pain in a subject is provided. The method comprises: a) providing a test cell population comprising cells capable of expressing one or more genes represented by nucleic acid sequences selected from the group consisting of SEQ ID NO: 1 to 64 or homologues or fragments thereof; b) contacting the test cell population with the test therapeutic agent; c) detecting the expression of one or more of the genes in the test cell population; d) comparing the expression of the gene(s) in the test cell population to the expression of the gene(s) in a reference cell population whose disease stage is known; and e) identifying a difference in expression levels of the considered sequences, if present, in the test cell population and the reference cell population, thereby identifying a therapeutic agent for treating pain.

In one embodiment, the pain is neuropathic pain.

According to a preferred embodiment of the invention, gene expression may be determined by PCR of cDNA, hybridization of a sample DNA, or by detecting the relevant protein.

In one aspect, the method of the present invention is envisioned in the development of a medicament to treat pain.

In one embodiment an isolated nucleic acid sequence selected from SEQ ID NO. 1 to 64 or homologues or fragments thereof or an isolated polypeptide encoded by one of these sequences is used as a medicament to treat pain. It is envisioned to be used alone or in a pharmaceutical composition.

A kit comprising one or more reagents for detecting one or more genes represented by nucleic acid sequences selected from the group consisting of SEQ ID NO. 1 to 64 or homologues or fragments thereof is also within the scope of the present invention.

A vector comprising any nucleic acid sequence selected from the group consisting of SEQ ID NO. 1 to 64 or homologues or fragments thereof is also within the scope of the present invention.

A host cell comprising such vector is also within the scope of the present invention.

Antibodies that selectively bind to a polypeptide encoded by a gene represented by a sequence selected from the group consisting of SEQ ID NO. 1 to 64 or homologues or fragments thereof are within the scope of the present invention. As are fragments, homologues, analogues and derivatives of such antibodies.

A transgenic animal wherein at least one of the sequences corresponding to the sequences represented by any of the SEQ ID NO: 1 to 64 is altered compared to the wild type is also within the scope of the present invention.

### BRIEF DESCRIPTION OF THE DRAWINGS

FIGS. 1 to 6 show comparison experiments of three pain models: the chronic constriction injury by the loose ligation of the sciatic nerve (CCI), the tight ligation of the partial sciatic nerve (PSL) and the tight ligation of spinal nerves (SNL). Expression levels of 6 differentially expressed sequences are represented. Each sequence is compared over a time period of 28 days to sham operated controls. Genes are described as differentially expressed when the sequence is up- or down-regulated at one time point in at least two of the three models. Over time the expression pattern can be determined as up-regulated in one to four time points; as down regulated in one to four time points or as mixed regulated if the type of regulation changes between up and down regulation at different time points.

X-axis describes the four time points analyzed by digital expression pattern display (DEPD), 1=day one post operation, 2=day 7 post operation, 3=day 14 post operation, 4=day 28 post operation. The Y-axis shows Ah which represents the normalized difference of expression (peak height) of a certain transcript between a control group and a treated group. x-fold difference in gene expression is calculated by

$$\frac{1 + Δ h}{1 - Δ h}$$

0=no change to control, +=up regulation, −=down regulation; (0.2=1.5 fold; 0.3=1.86 fold; 0.4=2.33 fold; 0.5=3 fold).

### DETAILED DESCRIPTION OF THE PREFERRED EMBODIMENT

In the following the models are designated as follows:

The model of Bennett is based on the chronic constriction injury by the loose ligation of the sciatic nerve. Therefore this model is designated as “CCI model”.

The model of Seltzer et al. is based on the tight ligation of the partial sciatic nerve and is therefore designated as “PSL model”.

The model of Kim & Chung is based on the tight ligation of spinal nerves and is therefore designated as “SNL model”.

These designations correspond to the designations used in the cited literature.

The three models are explained in more detail in the literature and in the examples below.

The term “polynucleotide sequence” or “nucleic acid sequence” designates in the present application any DNA or RNA sequence, independent of the length. Thus this term can describe short sequences like PCR primers or probes for hybridization, as well as whole genes or eDNA of these genes.

The term “polypeptide” or “amino acid sequence” designates a chain of amino acids, independent from their length, however, in any case more than one amino acid.

As “homologues” of polynucleotide sequences such polynucleotide sequences are designated which encodes the same type of protein as one of the polynucleotide sequences described herein. Accordingly as “homologues” of a polypeptide the polypeptides are designated which have an amino acid sequence, wherein at least 70%, preferably 80%, more preferably 90% of the amino acids are identical to one of the proteins of the present invention and wherein the replaced amino acids preferably are replaced by homologous amino acids. As “homologous” amino acids are designated which have similar features concerning hydrophobicity, charge, steric features, etc. Most preferred are amino acid sequences, containing the species- or family-dependent differences of the amino acid sequence. Particularly as “homologues” sequences are designated those which correspond to one of the cited sequences in another species or individual. For example if in the present invention a rat model is used and the cited polynucleotide sequence encodes the rat protein, the according polynucleotide sequence and protein of a mouse in a mouse model is designated as “homologue”. Further splice variants and members of gene families are designated as homologues.

“Fragments” of a polynucleotide sequence are all polynucleotide sequences, which have at least 10 identical base pairs compared to one of the polynucleotide sequences shown in the present application or by the genes represented by these polynucleotide sequences. The term “fragment” encloses therefore such fragments as primers for PCR, probes for hybridization, DNA fragments included in DNA vectors like plasmids, cosmids, BACs or viral constructs, as well as shortened splice variants of the genes identified herein. As a fragment of a protein (polypeptide) amino acid sequences are designated which have at least three amino acids, preferably at least 10 amino acids. Therefore fragments serving as antigens or epitopes are enclosed in this designation.

In the present application the term “sequence” is used when either a polynucleotide sequence (=nucleic acid sequence) or a polypeptide (=amino acid sequence) or a protein is meant. That means when it is irrelevant which type of sequence is used the type is not designated particularly, but with the more common term “sequence”.

The basis of the models and methods described in the present application is the examination and determination of the expression of genes, which are differentially expressed under pain or during development of pain. Therefore for the examination each sequence can be used which allows the determination of the expression rate of the considered gene. Such a sequence can be at least one of the polynucleotide sequences SEQ ID NO. 1 to 64 or homologues or fragments thereof, as well as the polypeptides encoded thereby, however, just as well polynucleotide sequences and the according polypeptides can be used which are (parts of) the genes represented by the polynucleotide sequences SEQ ID NO. 1 to 64.

According to the invention it has been found, that the genes represented by the polynucleotide sequences SEQ ID NO. 1 to 64 are differentially expressed correspondingly in one, two or all three different models for pain, which are the above described models “CCI”, “PSL”, SNL”.

Therefore the present invention provides sequences, which represent genes, which are differentially expressed under pain. Such polynucleotide sequences and the according polypeptides allow the determination and examination of pain development, conditions and progression. These sequences have not yet been regarded in relation to pain. For these examinations animal models can be used. As such a model any animal can be used wherein the necessary preparations can be carried out, however mammalian models are preferred. Even more preferred are rodents and lagomorpha, particularly preferred are rats, mice and rabbits. The most preferred animal model of the present invention is a rat model.

The sequences of the present invention further can be used for diagnosing a neuropathic pain status of a human outside of the living body by determining the expression levels of at least one of the cited sequences in comparison to the non-disease status. During treatment period of a patient the expression of the presently shown sequences can also be used for assessing the efficacy of pain treatment outside of the body. In this case blood, cerebrospinal fluid (CSF) or tissue is removed from the patient and expression is determined in the samples.

For determination and comparison of the expression levels of at least one of the genes identified in the present invention any of the commonly known methods can be used, either on RNA/cDNA level or on protein level. For example PCR, hybridization, micro array based methods, Western blot or 2-D protein gel analysis are suitable methods. One preferred method is the digital expression pattern display method (DEPD method), explained in detail in WO99/42610. The method used for determination of expression levels is not restrictive, as long as expressed amounts can be quantified.

The sequences of the present invention further can be used to develop new animal models for pain. By examination of the expression levels of at least one of the sequences shown it might be determined in several animals a procedure which is useful for generating a suitable animal model for different interesting conditions.

In such a newly generated animal model as well as in one of the known models the efficiency of compounds can be tested. Further as models for testing the efficiency of compounds assay systems can be used. Such assay systems may be in vivo, ex vivo or in vitro assays. In any case the models are contacted with the compound(s) to be tested and samples are obtained from these models, wherein expression levels of the sequences are determined and compared to the non-treated model.

Dependent from the used model the samples can be derived from whole blood, cerebrospinal fluid (CSF) or whole tissue, from cell populations isolated from tissue or blood or from single cell populations (i.e. cell lines).

In one embodiment of the invention cellular assays can be used. Preferred cells for cellular assays are eukaryotic cells, more preferably mammalian cells. Most preferred are neuronal-like cells, like SHSY5Y (neuroblastoma cell line), glial cell lines like TPH-1 and BV-2, astrocytic cell lines like U373MG and A7, or COS cells (African green monkey, kidney cells); CHO cells (Chinese hamster ovary), HEK-293 cells (human embryonic kidney).

Whereas the comparison of the expression levels (disease/non-disease status) of at least one of the provided sequences might give information about the examined disease status, it is preferred to determine the expression levels of more than one of the sequences simultaneously. Thus several combinations of the sequences can be used at different time points. By combination of several sequences a specific expression pattern can be determined indicating and/or identifying the conditions of the disease. The more expression rates are determined simultaneously, the more specific the result of the examination might be. However, good results can also be obtained by combination of only a few sequences. Therefore for the present invention it is preferred to compare the expression rates of at least two of the sequences provided herein, more preferred of at least 4, further more preferred of at least 6 of the sequences.

Since the presently provided sequences represent genes, which are differentially expressed, the expression rates of the single genes can be increased or decreased independently from each other. “Independently” in this context means that the expression rate of each of the genes can but need not be influenced by each other. In any case expression levels different from the non-disease status might be a hint to the disease status, which is examined.

The disease status, which is considered in the present invention is pain. The preferred types of pain are persistent/central pain, inflammatory pain (acute or chronic) or chronic pain. The most preferred type is neuropathic pain. Examples of such diseases are diabetic neuropathy, post-herpetic neuralgia, trigeminal neuralgia, cancer associated pain, spinal cord injury, multiple sclerosis, phantom pain, post-stroke pain, HIV associated pain, low back pain associated neuropathic pain, complex regional pain syndromes, like reflex sympathetic dystrophy and causalgia, myofacial syndromes or idiopathic pain conditions.

Independent of whether a pain status is diagnosed or characterized, a model for pain is characterized, the efficacy of pain treatment or the efficiency of a compound in a model shall be examined, the determination of the expression levels of at least one of the sequences is carried out outside of a living body. A method to obtain such results comprises:

a) providing a sample comprising cells or body fluids expressing or containing one or more genes or gene products represented by polynucleotide sequences selected from the group of SEQ ID NO. 1 to 64 or homologues or fragments thereof

b) detecting expression of one or more of the genes in said cells;

c) comparing the expression of the genes in the test cells to the expression of the same genes in reference cells whose expression stage is known, and

d) identifying a difference in expression levels of the considered sequences, if present, in the test cell population and the reference cell population.

As mentioned above, detection of the expression of the genes can be carried out by any method known in the art. The method of detection is not limiting the invention.

Expression levels can be detected either on basis of the polynucleotide sequences or by detecting the according polypeptide, encoded by said polynucleotide sequence.

Preferred methods for detection and determination of the gene expression levels are PCR of cDNA, generated by reverse transcription of expressed mRNA, hybridization of polynucleotides (Northern, Southern Blot systems, in situ hybridization), DNA-micro-array based technologies, detection of the according peptides or proteins via, e.g., Western Blot systems, 2-dimensional gel analysis, protein micro-array based technologies or quantitative assays like, e.g., ELISA tests.

The most preferred method for quantitative analysis of the expression levels is the differential expression pattern display method (DEPD), described in detail in WO99/42610.

The sequences of the present invention can further be used for identifying therapeutic agents and their efficiency for treating pain. For example a method can be used comprising:

a) providing a test cell population comprising cells capable of expressing one or more genes represented by nucleic acid sequences selected from the group consisting of SEQ ID NO: 1 to 64 or homologues or fragments thereof;

b) contacting said test cell population with the test therapeutic agent;

c) detecting the expression of one or more of the genes in said test cell population,

d) comparing the expression of the gene(s) in the test cell population to the expression of the gene(s) in a reference cell population whose disease stage is known; and

e) identifying a difference in expression levels of the considered sequences, if present, in the test cell population and the reference cell population, thereby identifying a therapeutic agent for treating pain.

Test cells can be obtained from a subject, an animal model or cell cultures of fresh cells or cell lines. Further in vitro assays may be used.

A method examining the different expression patterns of the differentially expressed genes therefore can be used for testing agents and compounds for their efficiency for treatment of pain. Which model is used is not relevant, as long as the model allows the determination of differences in expression amounts.

In such a model cells are contacted with the interesting agent or compound and expression of at least one of the genes considered in the present invention is determined in comparison to the expression of the same gene in cells which never have been contacted with the according agent/compound. Contacting the cells either can be effected by administering the agent/compound to an animal or by contacting isolated cells of tissue or blood or cells of cell lines in culture with the agent/compound.

By examination of the influence the considered agent/compound has on the expression of at least one of the genes the efficacy of the agent/compound can be estimated. This allows the decision whether it is worthwhile to develop a medicament containing such an agent or compound.

Whether the expression is determined on basis of mRNA generation or on basis of protein generation is not relevant, as long as the difference of the expression rate can be determined. Therefore the polynucleotide sequences, as well as the polypeptides or proteins shown in the present application can be used for the development of a medicament.

The development of a medicament can be desirable for example if the considered compound has an influence on the regulation of the expression rate or on the activity of any polynucleotide sequence or polypeptide of the present invention.

Said influence of a compound or agent can be examined by a method comprising contacting a sample comprising one of the nucleic acid sequences or of the polypeptides of the present invention with a compound that binds to said sequence in an amount sufficient to determine whether said compound modulates the activity of the polynucleotide or polypeptide sequence.

By such a method a compound or agent modulating the activity of any of the nucleic acid sequences or any polypeptides of the present invention can be determined.

Furthermore the sequences itself can be used as a medicament.

An example for such a use is the use of a polynucleotide sequence as an antisense agent. Antisense agents can hybridize to DNA or mRNA, inhibiting or decreasing transcription or translation, respectively. Thus, polynucleotide sequences of a gene, which is increased in expression rate under pain, can be used as antisense agents to decrease the expression rates of said gene. Further such polynucleotide sequences can be used for gene therapy.

Another example for such a use is the use of a polypeptide or a protein as a medicament. In case that the expression of a gene is decreased under pain and therefore not “enough” protein is provided by the body to maintain natural (healthy) conditions, said protein can be administered as a medicament.

A pharmaceutical composition comprising a polynucleotide sequence or a polypeptide according to the present invention can be any composition, which can serve as a pharmaceutical one. Salts or aids for stabilizing the sequences in the composition preferably are present.

For the determination of the expression of the relevant genes the generated sequences have to be detected. Therefore several reagents can be used, which are for example specific radioactive or non-radioactive (e.g., biotinylated or fluorescent) probes to detect nucleic acid sequences by hybridization, primer sets for the detection of one or several of the nucleic acid sequences by PCR, DNA microarrays, antibodies against one of the polypeptides, or epitopes, or antibody or protein microarrays. Such reagents can be combined in a kit, which can be sold for carrying out any of the described methods.

Further the sequences defined in the present invention can be used to “design” new transgenic animals as models for pain. Therefore the animals are “created” by manipulating the genes considered in the present application in a way that their expression in the transgenic animal differs from the expression of the same gene in the wild type. In which “direction” the gene expression has to be manipulated (up- or downregulation) depends on the gene expression shown in the present application. Methods of gene manipulation and methods for the preparation of transgenic animals are commonly known to those skilled in the art.

For further examinations or experiments it might be desirable to include any of the nucleic acids of the present invention into a vector or a host cell. By including the sequences in a host cell for example cellular assays can be developed, wherein the genes, polynucleotide sequences and the according proteins/polypeptides further can be used or examined. Such vectors, host cells and cellular assays therefore shall be considered as to fall under the scope of the present invention.

The following examples are provided for illustration and are not intended to limit the invention to the specific example provided.

### EXAMPLE 1

### Preparation of Rat Models

A) The CCI Model (Bennett & Xie 1988 Pain 33:87-107)

Nerve injury is created by loosely constrictive ligatures around the rat sciatic nerve (4 ligatures). The ligatures evoke intraneural edema, the swelling is opposed by the ligatures of the nerve strangulates. The constrictions remain for at least a month (hence, “chronic constriction injury”, CCI). It is known that the constriction injures nearly all of the nerves large myelinated axons; however, a variable but large percentage of the nerves unmyelinated axons remained intact. Although the nerve distal to the constriction is full of degeneration, the nerve proximal to the constriction appears normal, and there is no evidence of any primary afferent neuron dying. This model has a periphery that is innervated only by C-fibers and a greatly reduced number of A-delta fibers, and a spinal cord that is innervated by injured (but alive) A-beta low-threshold mechanoreceptors (AβLTMs), A-delta fibers (mostly injured, a few intact) and both injured and intact C-fiber afferents, many of which are nociceptors. This animal model provokes allodynia and hyperalgesia as well as spontaneous pain. Evidence of abnormal pain sensation is detected in the majority of CCI cases on the second PO day, and in nearly all cases by 5-7 days post injury. Abnormal pain sensation appears to reach peak severity in 10-14 days and to disappear in about 2 month when the pain is replaced by an apparently permanent state of hyperesthesia.

In detail, male Lewis rats (150-350 g body weight) were anesthetized with sodium pentobarbital (50 mg/kg i.p.). Thereafter the common sciatic nerve on the left side was exposed at the level of the middle of the thigh by blunt dissection through the biceps femoris. Proximal to the sciatics trifircation, about 7 mm of nerve was freed from adhering tissues and 4 ligatures (4.0 chromic gut) were tied loosely around it with 1 mm spacing. The length of nerve affected was 4-5 mm long. The desired degree of constriction retarded, but did not arrest, circulation through the superficial epineurial vasculature and sometimes produced a small, brief twitch in the muscle surrounding exposure. Control group animals were subjected to sham ligation (only left site), which represents the same operative procedure as the ligated one but without nerve ligation.

Four time points for gene expression profiling were chosen: 1 day, 7 days, 14 days, and 28 days post operation. Animals were tested for mechanical allodynia (von Frey test) one day before surgery and within 30-60 min before tissue preparation (dorsal root ganglia, spinal cord and thalamus). Tissues were frozen on liquid nitrogen prior to RNA preparation.

B) The PSL Model (Seltzer, et al. 1990 Pain 43: 205-218)

Disorders in nocifensive behavior following noxious and non-noxious stimuli begin hours after partial sciatic nerve injury and last for at least 7 months. This model is characterized by complex combination of rapid onset, allodynia to touch, hyperalgesia, mirror image phenomena, and dependence on the sympathetic outflow. This model resembles therefore many of the symptoms described for causalgia in man. The rapid initiation of these disorders and their contralateral appearance suggest central reorganization. This model may serve as a model for sympathetically maintained pain (SMP).

In detail, Lewis rats (male 150-350 g body weight) were anesthetized with sodium pentobarbital (50 mg/kg i.p.) followed by exposure of the left sciatic nerve at high-thigh level. Under 25× magnification, the dorsum of the nerve was carefully freed from surrounding tissues at a site near the trochanter just distal to the point at which the posterior biceps semitendinous (PBST) nerve branches off the common sciatic nerve. An 8-0 silicon-treated silk suture was inserted into the nerve with a ⅜ curved, reversed-cutting mini-needle, and tightly ligated so that the dorsal ⅓ to ½ of the nerve thickness was trapped in the ligature.

Control group animals were sham ligated, which represents the same operative procedure as the ligated one but without nerve ligation.

Four time points for gene expression profiling were chosen: 1 day, 7 days, 14 days, and 28 days post operation. Animals were tested for mechanical allodynia (von Frey test) one day before surgery and 30-60 min before tissue preparation (dorsal root ganglia, spinal cord and thalamus). Tissues were frozen on liquid nitrogen prior to RNA preparation.

C) The SNL Model (Kim & Chung 1992 Pain 50: 355-363)

In this model a tight ligation of the left LS and L6 spinal nerves or L5 spinal nerve alone is leading to a long-lasting hyperalgesia to noxious heat, at least 5 weeks of the affected foot. Long-lasting mechanical allodynia of the affected foot can be observed for at least 10 weeks. This model involves a complete ligation of spinal nerves L5 and L6 or only L5 (or L4), which is more reliable to other models where the number and types of ligated nerves are difficult to control,

In detail, male Lewis rats (150-350 g body weight) were anesthetized with sodium pentobarbital (50 mg/kg i.p.). Thereafter the sciatic spinal nerve ligation of the spinal nerve L5 was performed on the left site of the animals.

Control group animals were sham ligated (only left site), which represents the same operative procedure as the ligated one but without nerve ligation.

Four time points for gene expression profiling were chosen: 1 day, 7 days, 14 days, and 28 days post operation. Animals were tested for mechanical allodynia (von Frey test) one day before surgery and 30-60 min before tissue preparation (dorsal root ganglia, spinal cord and thalamus). Tissues were frozen on liquid nitrogen prior to RNA preparation.

### EXAMPLE 2

### Determination of Expression Levels

Gene expression profiling by DEPD-analysis starts with the isolation of 5-10 μg total RNA. In a second step, double-stranded cDNA is synthesized. Through an enzymatic digest of the cDNA with three different type IIS restriction enzymes, three pools with short DNA-fragments containing single-stranded overhangs are generated. Afterwards, specific DNA-adaptor-molecules are ligated and in two subsequent steps 3,072 PCR reactions are performed by using 1024 different unlabelled 5′ primers and a common FAM-fluorescent-labelled 3′-primer in the last PCR step. Subsequently, the 3072 PCR pools are analyzed on an automatic capillary electrophoresis sequencer.

Differential gene expression pattern of single fragments are determined by comparison of normalized chromatogram peaks from the control groups and corresponding operated animals.

### EXAMPLE 3

### Sequencing and Databank Analysis of the Obtained Sequences

Differentially expressed peaks are confirmed on polyacrylamide gels by using radioactive labelled 3′ primer instead of the FAM-fluorescent primer. Differentially expressed bands are cut from the gel. After a short elution step in 60 μl 10 mM Tris pH 8, fragments are re-amplified by PCR using the same primer as used in the DEPD analysis. Resulting PCR products are treated with a mixture of Exonuclease I and shrimp alkaline phosphatase prior to direct sequencing. Sequencing reactions are performed by using DYEnamic-ET-dye terminator sequencing kit (Amersham) and subsequently analyzed by capillary electrophoresis (Megabace 1000, Amersham).

Prior to a BLAST sequence analysis (Altschul et al. 1997 Nucleic Acids Res 25:3389-3402) against Genbank (1^{st} annotation, Table 1) and Unigene (2^{nd} annotation, Table 1), all sequences are quality verified and redundant sequences or repetitive motifs are masked.

Results are shown in Table 1.

| Seq | | | Fragment |
|---|---|---|---|
| ID | Accession | | length |
| NO | number | Name | [bp] |
| 1 | | novel | 250 |
| 2 | Y00054 | HSC73 | 244 |
| 3 | BE112971 | UI-R-BJ1-awa-h-11-0-UI.s1 UI-R-BJ1 | 155 |
| | | Rattus norvegicus cDNA clone UI-R- | |
| | | BJ1-awa-h-11-0-UI 3′, mRNA sequence | |
| 4 | BE928419 | RC0-CT0499-290800-024-c01 CT0499 | 132 |
| | | Homo sapiens cDNA, mRNA sequence | |
| 5 | AB030215 | COXII or Elf-1 (might be 3′ similarity) | 85 |
| | | Rattus norvegicus mRNA for transcrip- | |
| | | tion factor Elf-1, complete cds. Janu- | |
| | | ary 2001 | |
| 6 | BF288590 | EST453181 Rat Gene Index, normal- | 100 |
| | | ized rat, Rattus norvegicus cDNA | |
| | | Rattus norvegicus cDNA clone | |
| | | RGIGV25, mRNA sequence. | |
| 7 | BF288184 | EST452775 Rat Gene Index, normal- | 178 |
| | | ized rat, Rattus norvegicus cDNA | |
| | | Rattus norvegicus cDNA clone | |
| | | RGIGQ58 3′ sequence | |
| 8 | | novel | 85 |
| 9 | AW534512 | UI-R-BS0-ans-d-12-0-UI.s1 UI-R-BS0 | 167 |
| | | Rattus norvegicus cDNA clone UI-R- | |
| | | BS0-ans-d-12-0-UI 3′, mRNA sequence. | |
| 10 | M55424 | neurofilament NF-L | 150 |
| 11 | AB023781 | cathepsin Y | 255 |
| 12 | X61479 | CSF-1 receptor | 158 |
| 13 | AJ001633 | Mus musculus mRNA for annexin III | 116 |
| 14 | M88347 | Rat pseudo-cystathionine beta-synthase | 325 |
| | | mRNA, complete cds (type 4 of four | |
| | | alternatively spliced mRNAs). | |
| | | April 1993 | |
| 15 | AI058239 | EST49 Rat cochlea outer hair cells | 97 |
| | | Lambda Zap Express Library Rattus | |
| | | norvegicus cDNA clone 300c 5′, mRNA | |
| | | sequence. March 1999 | |
| 16 | BF286224 | EST450815 Rat Gene Index, normal- | 76 |
| | | ized rat, Rattus norvegicus cDNA | |
| | | Rattus norvegicus cDNA clone | |
| | | RGIFN38 5′ sequence | |
| 17 | BF807839 | RC3-CI0042-111100-011-f06 CI0042 | 167 |
| | | Homo sapiens cDNA, mRNA sequence | |
| 18 | AW531794 | UI-R-C4-akz-b-06-0-UI.s1 UI-R-C4 | 181 |
| | | Rattus norvegicus cDNA clone UI-R- | |
| | | C4-akz-b-06-0-UI 3′, mRNA sequence. | |
| 19 | | novel | 153 |
| 20 | X83231 | R. norvegicus mRNA for pre-alpha- | 97 |
| | | inhibitor, heavy chain 3 | |
| 21 | M72414 | MAP4 | 138 |
| 22 | V01227 | alpha tubulin | 209 |
| 23 | AK014144 | 1P: Mus musculus 13 days embryo head | 359 |
| | | cDNA, RIKEN full-length enriched li- | |
| | | brary, clone: 3110038L02, full insert | |
| | | sequence. July 2001 | |
| 24 | AK004964 | Mus musculus adult male liver cDNA, | 167 |
| | | RIKEN full-length enriched library, | |
| | | clone: 1300011D16, full insert se- | |
| | | quence. July 2001 Weakly similar to | |
| | | T20253 hypothetical protein F53E4.1 - | |
| | | Caenorhabditis elegans [C. elegans] | |
| | | (unigene) | |
| 25 | BE101201 | UI-R-BJ1-aua-c-03-0-UI.s1 UI-R-BJ1 | 121 |
| | | Rattus norvegicus cDNA clone UI-R- | |
| | | BJ1-aua-c-03-0-UI 3′, mRNA sequence. | |
| | | June 2000 Weakly similar to T21321 | |
| | | hypothetical protein F25B3.3 - | |
| | | Caenorhabditis elegans [C. elegans] | |
| | | (unigene) | |
| 26 | AF140232 | Rattus norvegicus calcium binding | 246 |
| | | protein (S100A6), calcyclin | |
| 27 | | novel | 113 |
| 28 | AY004290 | Rattus norvegicus scg10-like-protein | 254 |
| 29 | AI706278 | UI-R-AC0-yj-c-03-0-UI.s1 UI-R-AC0 | 135 |
| | | Rattus norvegicus cDNA clone UI-R- | |
| | | AC0-yj-c-03-0-UI 3′, mRNA sequence. | |
| | | June 1999 | |
| | | ubiquitin carboxy-terminal hydrolase | |
| | | L1 (unigene) | |
| 30 | X62322 | R. norvegicus mRNA for | 462 |
| | | epithelin 1 and 2. August 1992 | |
| 31 | X16417 | Rat mRNA for beta-globin. Septem- | 208 |
| | | ber 1993 | |
| 32 | | novel | 211 |
| 33 | AI044283 | UI-R-C1-kb-f-04-0-UI.s2 UI-R-C1 | 190 |
| | | Rattus norvegicus cDNA clone UI-R- | |
| | | C1-kb-f-04-0-UI 3′, mRNA sequence. | |
| | | July 1999 Weakly similar to T46402 | |
| | | hypothetical protein | |
| | | DKFZp434H2121.1 [H. sapiens] | |
| | | (unigene) | |
| 34 | S74324 | clone E501/543/588/5105, estrogen | 82 |
| | | induced gene, rats, Sprague-Dawley, | |
| | | hypothalamus, mRNA Partial, 252 nt]. | |
| | | April 1995 | |
| 35 | BF410973 | UI-R-CN0-bmi-b-12-0-UI.s1 UI-R- | 164 |
| | | CN0 Rattus norvegicus cDNA clone | |
| | | UI-R-CN0-bmi-b-12-0-UI 3′, mRNA | |
| | | sequence. November 2000 | |
| 36 | | novel | 247 |
| 37 | M31038 | Rat MHC class I non-RT1.A alpha-1- | 233 |
| | | chain mRNA, complete cds. April 1993 | |
| 38 | BF288184 | EST452775 Rat Gene Index, normal- | 118 |
| | | ized rat, Rattus norvegicus cDNA | |
| | | Rattus norvegicus cDNA clone | |
| | | RGIGQ58 3′ sequence, mRNA se- | |
| | | quence. November 2000 | |
| 39 | AI058239 | EST49 Rat cochlea outer hair cells | 118 |
| | | Lambda Zap Express Library Rattus | |
| | | norvegicus cDNA clone 300c 5′, mRNA | |
| | | sequence. March 1999 | |
| 40 | D45249 | Rat mRNA for proteasome activator | 299 |
| | | rPA28 subunit alpha, complete cds. | |
| | | February 1999 | |
| 41 | | novel | 112 |
| 42 | M17083 | Rat major alpha-globin mRNA, com- | 336 |
| | | plete cds. April 1993 | |
| 43 | | novel | 191 |
| 44 | BF290323 | EST454914 Rat Gene Index, normal- | 129 |
| | | ized rat, Rattus norvegicus cDNA | |
| | | Rattus norvegicus cDNA clone | |
| | | RGIHV04 3′ sequence, mRNA se- | |
| | | quence. November 2000 | |
| 45 | S56463 | HKII = hexokinase II [rats, epididymal | 230 |
| | | fat pad, mRNA Partial, 266 nt, seg- | |
| | | ment 1 of 2]. June 1993 | |
| 46 | | novel | 119 |
| 47 | | novel | 122 |
| 48 | L22643 | Rat anti-acetylcholine receptor antibody | 301 |
| | | gene, kappa-chain, VJC region, com- | |
| | | plete cds. July 1996 | |
| 49 | AF028784 | Rattus norvegicus glial fibrillary acidic | 473 |
| | | proteins alpha and delta (GFAP) gene, | |
| | | alternatively spliced products, complete | |
| | | cds. June 1999 | |
| 50 | | novel | 216 |
| 51 | BF557085 | UI-R-E1-go-a-09-0-UI.r1 UI-R-E1 | 446 |
| | HSA292757 | Rattus norvegicus cDNA clone UI-R- | |
| | | E1-go-a-09-0-UI 5′, mRNA sequence. | |
| | | December 2000 Moderately similar to | |
| | | TBB1 RAT TUBULIN BETA CHAIN | |
| | | [R. norvegicus] (unigene) | |
| 52 | AB010743 | Rattus norvegicus mRNA for UCP2, | 305 |
| | | complete cds. February 1999 | |
| 53 | X82396 | R. norvegicus mRNA for cathepsin B. | 420 |
| | | September 1996 | |
| 54 | Z12298 | R. norvegicus mRNA for dermatan | 205 |
| | | sulfate proteoglycan-II (decorin). | |
| | | February 1997 | |
| 55 | D90035 | Rattus norvegicus mRNA for | 142 |
| | | calcineurin A alpha, complete cds. | |
| | | July 1999 | |
| 56 | D83349 | Rat mRNA for short type PB-cadherin, | 150 |
| | | complete cds. February 1999 | |
| 57 | X74125 | R. norvegicus mRNA for NAD+- | 298 |
| | | isocitrate dehydrogenase, gamma sub- | |
| | | unit. July 1995 | |
| 58 | BF407932 | UI-R-BJ0p-ait-h-01-0-UI.s2 UI-R-BJ0p | 338 |
| | | Rattus norvegicus cDNA clone UI- | |
| | | R-BJ0p-ait-h-01-0-UI 3′, mRNA se- | |
| | | quence. November 2000 | |
| 59 | M18053 | Rat ferritin heavy subunit gene | 151 |
| 60 | | novel | 300 |
| 61 | | novel | 125 |
| 62 | Z22593 | M. musculus fibrillarin mRNA. | 204 |
| 63 | AI058239 | EST49 Rat cochlea outer hair cells | 111 |
| | | Lambda Zap Express Library Rattus | |
| | | norvegicus cDNA clone 300c 5′, mRNA | |
| | | sequence. March 1999 | |
| 64 | AF093567 | 1P: Rattus norvegicus myocilin mRNA, | 150 |
| | | complete cds. August 1999 (Myoc/tigr) | |

### EXAMPLE 4

### Comparison of Differentially Expressed Sequences in Three Model Systems

Four time points for gene expression profiling were chosen for all three models: 1 day, 7 days, 14 days, and 28 days post operation. Behavioral testing of the animals was performed one day before operation and 30-60 min prior to the tissue preparation. After DEPD analysis differentially expressed peaks obtained for the three different pain models were compared to identify overlapping gene expression patterns.

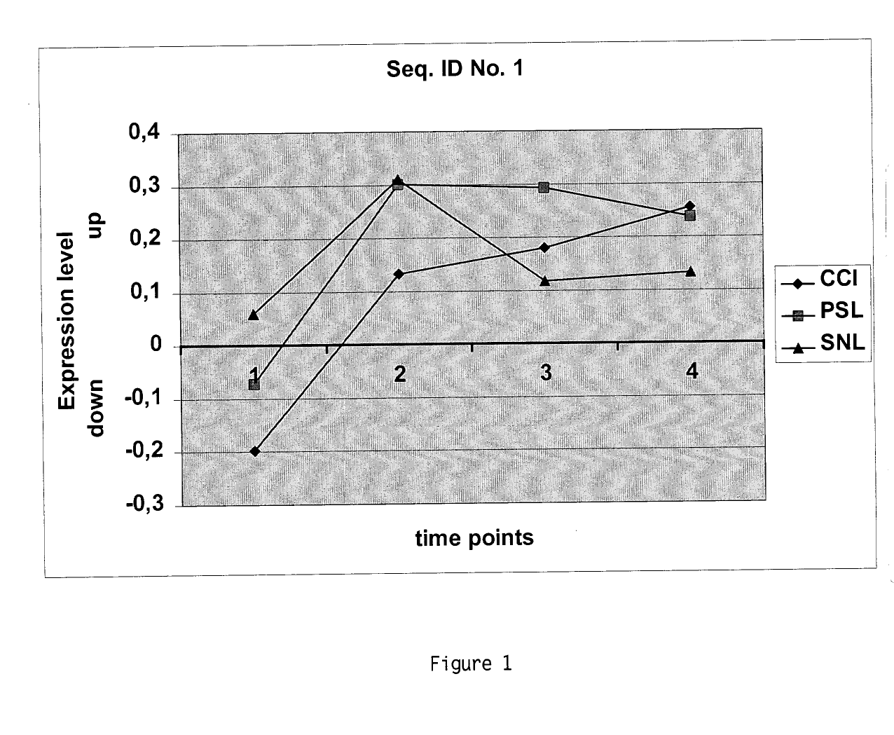

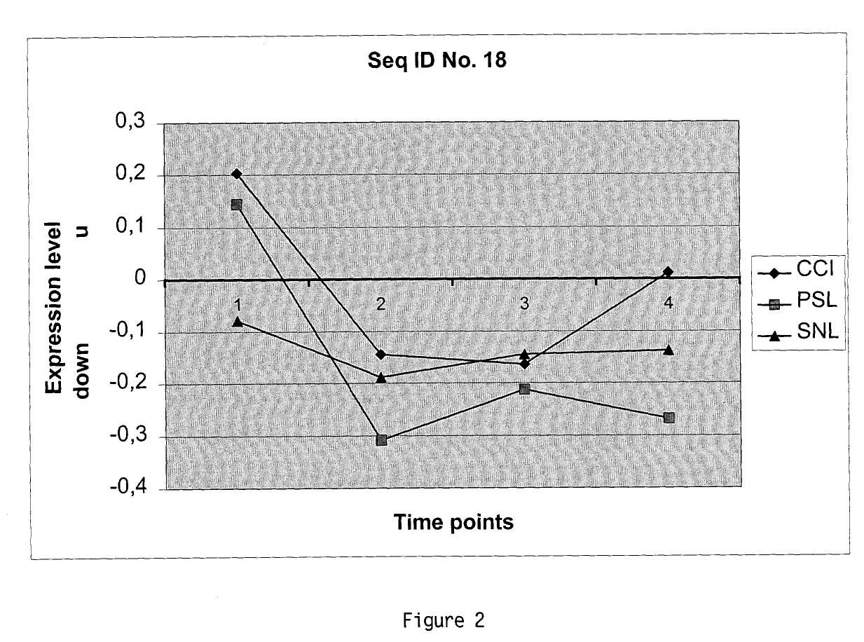

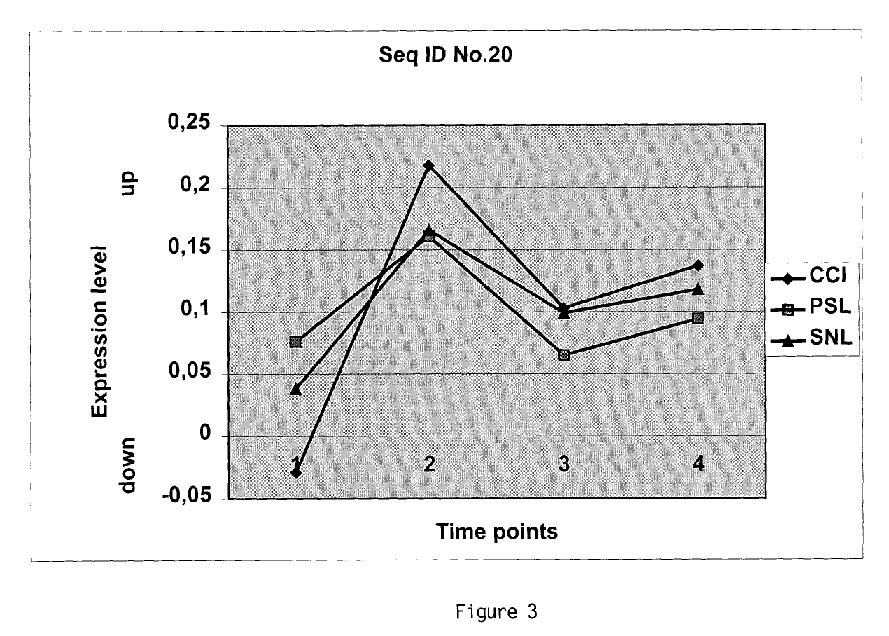

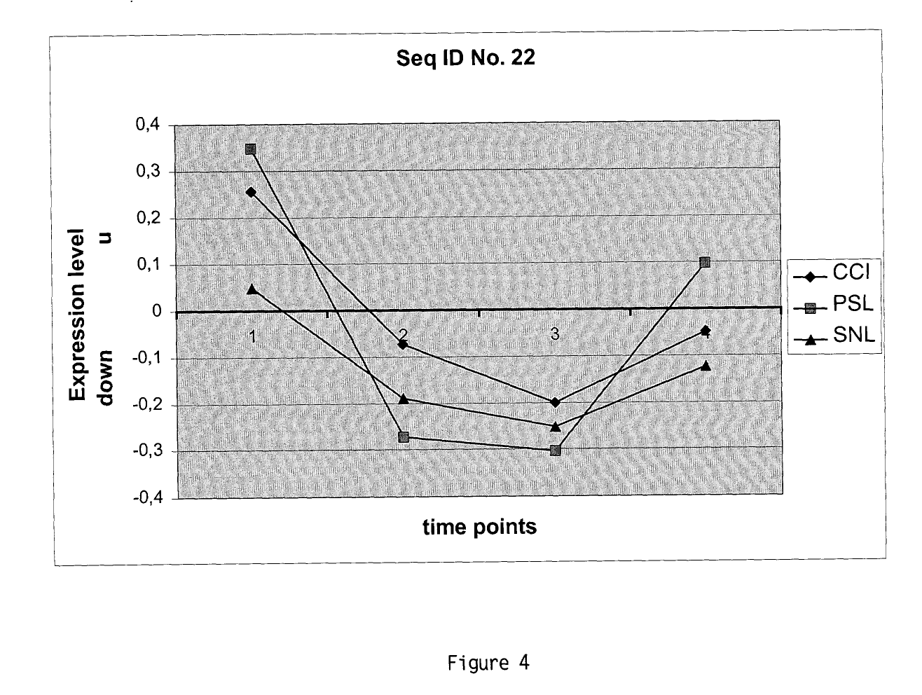

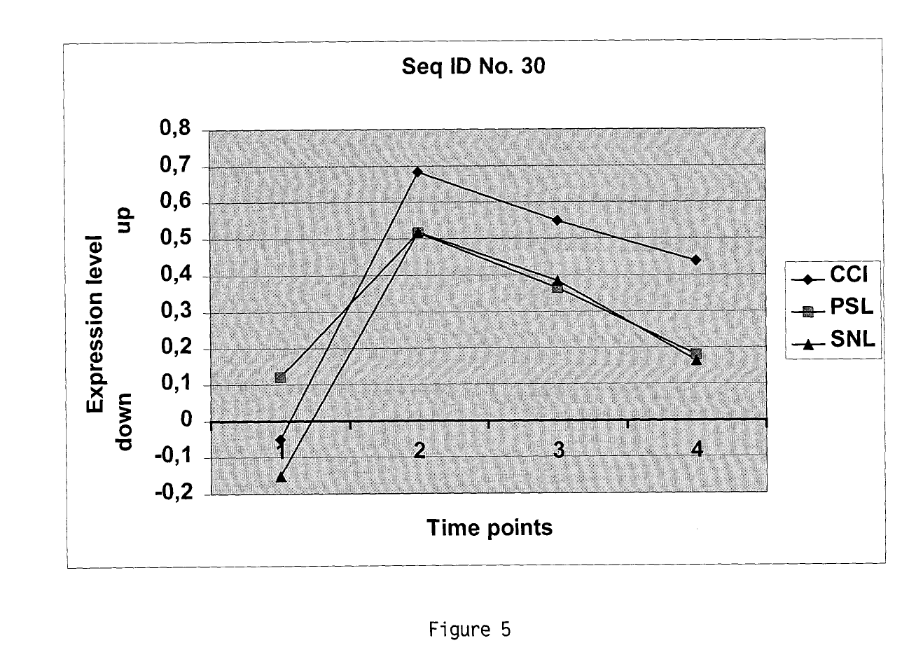

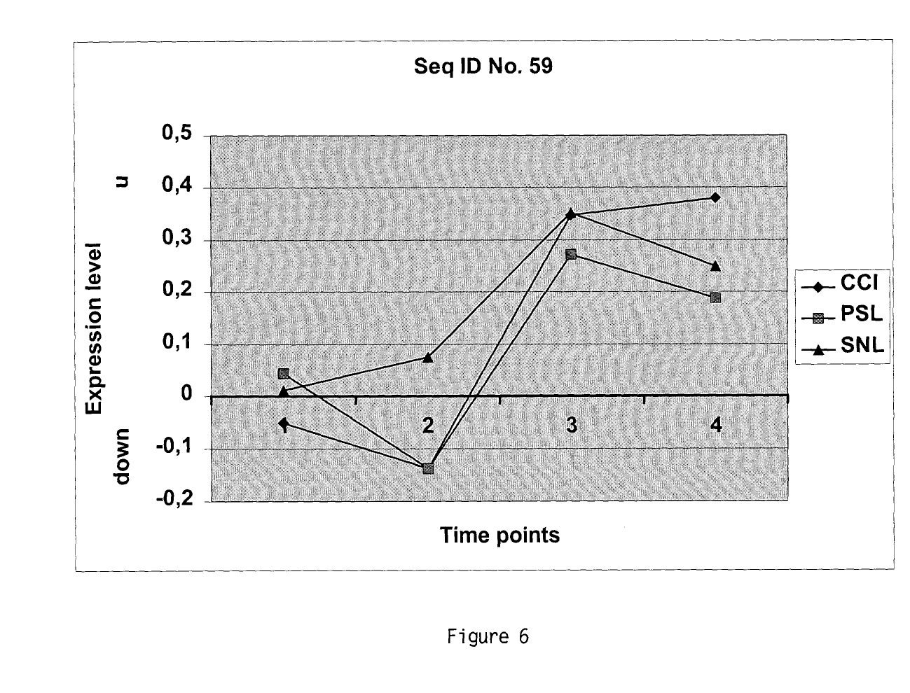

Results are shown in Table 2. For 6 selected sequences the regulation is further shown in FIGS. 1 to 6 (Regulation of Seq No: 1, 18, 20, 22, 30 and 59).

| Seq ID No. | Regulation |
|---|---|
| 1 | up |
| 2 | up |
| 3 | up |
| 4 | down |
| 5 | mixed |
| 6 | up |
| 7 | down |
| 8 | up |
| 9 | mixed |
| 10 | down |
| 11 | up |
| 12 | up |
| 13 | up |
| 14 | up |
| 15 | down |
| 16 | mixed |
| 17 | up |
| 18 | down |
| 19 | down |
| 20 | up |
| 21 | up |
| 22 | mixed |
| 23 | down |
| 24 | down |
| 25 | up |
| 26 | mixed |
| 27 | mixed |
| 28 | mixed |
| 29 | mixed |
| 30 | up |
| 31 | mixed |
| 32 | mixed |
| 33 | up |
| 34 | up |
| 35 | up |
| 36 | up |
| 37 | up |
| 38 | down |
| 39 | mixed |
| 40 | up |
| 41 | down |
| 42 | up |
| 43 | mixed |
| 44 | mixed |
| 45 | up |
| 46 | down |
| 47 | up |
| 48 | up |
| 49 | up |
| 50 | down |
| 51 | mixed |
| 52 | up |
| 53 | up |
| 54 | mixed |
| 55 | up |
| 56 | mixed |
| 57 | mixed, |
| 58 | down |
| 59 | up |
| 60 | up |
| 61 | up |
| 62 | up |
| 63 | mixed |
| 64 | up |

For each DNA fragment, gene expression patterns obtained in the three pain models were matched. “Up”, “down”, and “mixed” regulation is defined as overlapping pattern in at least two of the three models at one or more time points.

What is claimed is:

1. A method for characterizing a pain status, comprising:
a) detecting a level of expression of at least four gene sequences selected from the group consisting of SEQ ID NO. 1 to 64 or homologues or fragments thereof, in a test sample from an individual with pain;
b) detecting a level of expression of said at least four gene sequences in a control sample; and
c) comparing the levels of expression in the test and control samples, thereby characterizing the pain status.

2. The method of claim 1, wherein the control sample is from an individual without pain.

3. The method of claim 1, wherein the control sample is derived from a cell line.

4. The method of claim 1, wherein said individual is an animal.

5. The method of claim 1, wherein said individual is a human.

6. The method of claim 2, wherein said individual is an animal.

7. The method of claim 2, wherein said individual is a human.

8. The method of claim 1, wherein the test sample and control sample are derived independently from a source selected from the group consisting of whole tissues, blood, cerebrospinal fluid, isolated cells, and cell lines.

9. The method of claim 1, wherein the test sample and control sample are derived independently from an in vivo sample, an in vitro sample, or an ex vivo sample.

10. The method of claim 1, wherein the levels of expression in the test sample are increased relative to the levels of expression in the control sample.

11. The method of claim 1, wherein the levels of expression in the test sample are decreased relative to the levels of expression in the control sample.

12. The method of claim 1, further comprising detecting a level of expression of said at least four gene sequences in the test sample prior to and following administration of a pain treatment; and comparing the levels of expression prior to and following administration, thereby assessing the efficacy of the pain treatment.

13. The method of claim 1, wherein said individual is an animal used in an animal model for studying pain.

14. The method of claim 13, wherein the animal is subjected to a pain stimulus.

15. The method of claim 14, wherein the animal is administered a compound that may alter the pain status.

16. The method of claim 1, further comprising detecting at least six gene sequences selected from the group consisting of SEQ ID NO. 1 to 64 or homologues or fragments thereof, in the test sample and the control sample.

17. The method of claim 1, further comprising detecting at least eight gene sequences selected from the group consisting of SEQ ID NO. 1 to 64 or homologues or fragments thereof, in the test sample and the control sample.

18. The method of claim 1, further comprising detecting at least ten gene sequences selected from the group consisting of SEQ ID NO. 1 to 64 or homologues or fragments thereof, in the test sample and the control sample.

19. The method of claim 1, further comprising detecting at least twelve gene sequences selected from the group consisting of SEQ ID NO. 1 to 64 or homologues or fragments thereof, in the test sample and the control sample.

20. The method of claim 1, wherein said individual with pain is suffering from neuropathic pain.

21. The method of claim 1, wherein detecting the level of expression in the test sample and control sample further comprises at least one method selected from the group consisting of PCR of a cDNA, hybridization of a sample DNA, and detecting one or more polypeptides encoded by said at least four gene sequences or homologues or fragments thereof.

22. A method for characterizing a pain status, comprising:
a) providing a test sample comprising a cell or a body fluid expressing a polynucleotide sequence selected from the group consisting of SEQ ID NO. 1 to 64 or homologues or fragments thereof;
b) detecting expression of said polynucleotide in said test sample;
c) comparing the expression of said polynucleotide in said test sample to expression of the same polynucleotide in a reference sample whose expression stage is known; and
d) identifying a difference in the levels of expression between said test sample and said reference sample, thereby characterizing the pain status.

23. A method for identifying a therapeutic agent for treating pain in a subject, comprising:
a) providing a test cell capable of expressing a polynucleotide sequence selected from the group consisting of SEQ ID NO: 1 to 64 or homologues or fragments thereof;
b) detecting expression of said polynucleotide sequence in said test cell;
c) contacting said test cell with the therapeutic agent;
d) detecting expression of said polynucleotide sequence in said test cell contacted with the therapeutic agent;
e) comparing the expression of said polynucleotide sequence in step (b) to the expression of said polynucleotide sequence in step (d); and
f) identifying a change in expression of said polynucleotide in the presence of the therapeutic agent, thereby identifying the therapeutic agent for treating pain.

24. The method of claim 17, wherein said pain is neuropathic pain.

25. The method of claim 17, wherein detecting expression of said polynucleotide further comprises at least one method selected from the group consisting of PCR of a cDNA, hybridization of a sample DNA, and detecting a polypeptide encoded by said polynucleotide or homologue or fragment thereof.

26. A pharmaceutical composition, comprising a polynucleotide selected from the group consisting of SEQ ID NO: 1 to 64 or homologues or fragments thereof, or a polypeptide encoded by said polynucleotide.

27. A kit comprising a reagent for detecting a polynucleotide selected from the group consisting of SEQ ID NO: 1 to 64 or homologues or fragments thereof.

28. A vector, comprising a polynucleotide selected from the group consisting of SEQ ID NO: 1 to 64 or homologues or fragments thereof.

29. A host cell, comprising a polynucleotide selected from the group consisting of SEQ ID NO: 1 to 64 or homologues or fragments thereof.

30. An antibody that selectively binds to a polypeptide encoded by a polynucleotide selected from the group consisting of SEQ ID NO: 1 to 64 or homologues or fragments thereof.

31. A transgenic animal, comprising a polynucleotide selected from the group consisting of SEQ ID NO: 1 to 64 or homologues or fragments thereof, wherein said polynucleotide has been altered compared to a wild type phenotype.
